(19) United States
(12) Patent Application Publication
(10) Pub. No.: US 2004/0214897 A1
(43) Pub. Date: Oct. 28, 2004

(54) Uses for 2-amino-2-propane-1,3-diols

(76) Inventors: Carole Welsch, Singapore (SG); Rao Movva, Binningen (CH)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/484,570

(22) PCT Filed: Jul. 22, 2002
(86) PCT No.: PCT/EP02/08164

(30) Foreign Application Priority Data
Jul. 23, 2001 (GB) 0117921.7

(51) Int. Cl. A61K 31/137

(52) U.S. Cl. 514/649

(57) ABSTRACT

Disclosed is the identification of specific molecular targets and cellular pathways involved in the mechanism of action of 2-amino-2-propane-1;3-diols.

The present invention relates to the inhibition of yeast growth and to the identification of the specific molecular targets and cellular pathways involved in the mechanism of action of a group of 2-amino-2-propane-1,3-diols.

Suitable 2-amino-2-propane-1,3-diols include e.g. compounds of formula I

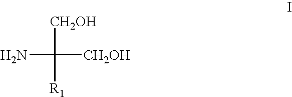

wherein R₁ is 2-(4-octylphenyl)ethyl or 2-[4-(1-oxo-5-phenylpentyl)phenyl]ethyl, or a pharmaceutically acceptable salt thereof.

Examples of pharmaceutically acceptable salts of the compounds of the formula I include salts with inorganic acids, such as hydrochloride, hydrobromide and sulfate, salts with organic acids, such as acetate, fumarate, maleate, benzoate, citrate, malate, methanesulfonate and benzenesulfonate salts, or, when appropriate, salts with metals such as sodium, potassium, calcium and aluminium, salts with amines, such as triethylamine and salts with dibasic amino acids, such as lysine. The compounds and salts of formula I encompass hydrate and solvate forms.

2-Amino-2-[2-(4-octylphenyl)ethyl]propane-1,3-diol (“FTY720”) and 2-amino-2-{2-[4-(1-oxo-5-phenylpentyl)phenyl]ethyl}propane-1,3-diol (“Compound Y”) in free form or in a pharmaceutically acceptable salt form, e.g. hydrochloride, are compounds having immunosuppressive properties and are analogues of myriocine, a product from Isaria sinclairii. They prolong the survival of allografts as well as prevent the development of pathology in GvHD and autoimmune type I diabetes. The mode of immunosuppressive action of these 2 compounds is completely different from those of the other established immunosuppressive compounds such as cyclosporin A (CsA), FK506 and rapamycin that block T cell activation by interfering with signal transduction. CsA and FK506 inhibit the expression of genes required for T cell response to antigen presented on antigen presenting cells, including IL-2. Rapamycin inhibits the response of T cells to IL-2. FTY720 or Compound Y induces the rapid and marked reduction of peripheral blood lymphocytes (PBL) in vivo. The immunosuppressants CsA, FK506 and rapamycin potently inhibit the mammalian immune system as well as the growth of several yeast strains. Studies on the anti-fungal effects of these compounds have revealed that both the mechanisms of drug action and the drug targets themselves are highly conserved from these unicellular eukaryotes to humans.

It has been reported that the number of lymphocytes are increased in peripheral lymph nodes (PLN), mesenteric lymph nodes (MLN) and Peyer's patches (PP), suggesting that accelerated lymphocyte homing Into lymph nodes (LN) from peripheral blood is a main mode of action for immunosuppressive effects of FTY720 or Compound Y. Besides, it has been reported that long-term administration of FTY720 not only reduces the number of PBL, but also inhibits T cell functions. However, the precise molecular mechanism of FTY720 or Compound Y is still unknown and the elucidation of direct target molecules and pathways can provide very important information to understand the immunosuppressive and side effect profile of these molecules as well as novel targets for future drug discovery.

It has now been observed that FTY720 or Compound Y exhibits an antifungal activity on Saccharomyces Cerevisie an unicellular eukaryotic organism of Saccharomyces genus. More particularly, it has been found that FTY720 or Compound Y interferes with the nutrient transport pathways, e.g. amino acids such as tryptophan and leucine in this model unicellular eukaryote and ubiquitin protein degradation pathway and targets are Involved in its mechanism of action.

Additionally, it was found that the ubiquitin pathway and targets therein have significant role in the growth inhibitory effects of FTY720 or Compound Y.

The new activity of the compounds of formula I, e.g. FTY720, is demonstrated in e.g. in vitro assay, as follows:

The compound, e.g. FTY720, is dissolved in distilled water to be 10 mM, the resulting stock solution is kept at 4° C. and diluted to the appropriated concentrations with medium used for yeast culture. Standard laboratory yeast strain in this case derivatives of JK9-3d (a, alpha and diploid) are plated on to media containing the varying concentrations of the compound to be tested and their growth are compared to that in medium without the compound. Cultivation and maintenance of cells are carried out in YEPD medium (Difco) or synthetic medium (SD).

The growth of both yeast strains JK9-3dα, MH272-1da and MH272-1da/α is inhibited by a compound of formula I, e.g. FTY720, at 30 μM in the YPED solid medium at 30° C. or in minimal medium at 19° C.

Isolation of FTY720 Resistance-Conferring Genes by Over-Expression

If the growth sensitivity of these yeast strains is due to inhibition of specific functional protein targets in the cells, such inhibition could be relieved by overexpressing such a function through introduction of multiple genetic copies of such function. Accordingly the FTY720 sensitive strain JK9-3dα has been transformed with yeast genomic DNA libraries cloned in high copy plasmid (2 μm) URA3 selectable using Yeast Transformation System (Clontech). FTY720-resistant transformants are selected on synthetic medium lacking uracil in the presence of 30 μM of FTY720. The total number of URA3+transformants is >20,000. The first selection Is performed at 19° C. because even the parent yeast strain JK9-3dα is resistant to FTY720 in synthetic medium at 30° C. After the first selection for resistance, FTY720-resistant growth phenotype of transformants is reconfirmed by restreaking individual colonies on to medium with the compound. Plasmid which conferred resistance to FTY720 are recovered according to standard methods, amplified in E. coli and reintroduced into JK9-3da for the confirmation of FTY720 resistance. The DNA fragments which conferred FTY720 resistance are isolated to determine sequence of both terminii of insert DNA and the DNA sequence is compared with Saccharomyces Genome Database. Then, the responsible single genes for the FTY720 resistance are further identified from the DNA fragments due to their common presence in different fragments and by using specific restriction enzymes to subclone them into yeast plasmid vector as shorter fragments (Genes Vap1, UBP5, UBP11 and Caj1 are identified by this method).

Isolation of FTY720 Resistant Genomic Mutants

FTY720-resistant genomic mutants are isolated from JK9-3da, MH272a or MH272a/α by treatment with 3% ethyl methane sulphonate (EMS) for 10-20 min. as well as spontaneous mutants without treatment of mutagen. The survival ratio after EMS treatment is 48-67%. Yeast cells treated with or without EMS are cultured in the YEPD solid medium in the presence of 30 μM of FTY720 and FTY720-resistant colonies are selected. The first selection is performed at 19° C. in order to reduce pseudo positive colonies and the resistance to FTY720 is confirmed at 30° C. These genomic mutants are classified as dominant or recessive mutations according to standard methods of yeast genetics. Briefly yeast strains can be constructed to be of diploid or haploid nature using mating and dissection technologies. Diploid strains contain two copies of the genome and haploid strains contain one copy of the genome. A given mutation then is defined as dominant if its function dominates that of unmodified function of the same gene when both are placed in the diploid strain. Conversely the observed phenotype of the recessive mutation is suppressed by its wild type counterpart in a diploid background. Accordingly the genes conferring such dominant or recessive phenotypes can be cloned by complementation into the corresponding wild type strains. A recessive mutant can be identified by the cloning in and complementation of the corresponding wild type gene through a plasmid library and a dominant gene can be cloned by direct selection of the gene in a wild type background. Gene GNP1 is identifed as dominant mutant and BUL1 is identified as recessive mutant.

Isolation of Yeast Genes Conferring the FTY720 Resistance by Multiple Copies

Four kinds of plasmid DNA are found to overcome the growth-inhibitory effects of FTY720 by transformation of FTY720 sensitive wild-type yeast JK9-3da with genomic DNA library constructed in high (2 μm)-copy-number plasmid. From each fragment or resistance plasmid, single responsible genes, which overcome the anti-fungal effects of this compound at the increased copy number, are identified by subcloning using restriction enzymes. One is amino acid transporter VAP1, the others are a putative deubiquitinating enzyme UBP11 and UBP5. The last one is CAJ1 (heat shock protein). All of them overcome the growth-inhibitory effects of 30 μM of FTY720 in the high-copy-number plasmid.

General Isolation of Mutants

In order to identify other genes/proteins involved in anti-fungal effects of FTY720, genomic mutants resistant to 30 μM, at least, of FTY720 are isolated from haploid (JK9-3dα and MH272-1 da) or diploid yeast strain (MH272-1 da/α) by treatment with or without 3% EMS. The total number of FTY720-resistant mutants isolated in this study is 13 spontaneous (JK9-3dα: 6, MH272-1 da: 6, MH272-1 da/α: 1) and 37 EMS-treated ones (JK9-3dα: 10, MH272-1da: 17, MH272-1 da/α: 10). First of all, those mutants are characterized by genetic crosses to wild-type FTY720-sensitive yeast and classified into 23 dominant (spontaneous: 3, EMS treated: 20), 22 recessive (spontaneous 10, EMS treated: 12) or 5 intermediate ones (spontaneous: 0, EMS treated: 5). From these 23 dominant mutants, 6 genomic DNA libraries is constructed in low-copy number plasmid pRS416 from 2 diploid (one spontaneous and one EMS-treated) and 4 haploid dominant mutants (one spontaneous and three EMS-treated) as well as from wild-type JK9-3dα, and they are used to identify mutation which confer FTY720 resistance.

The glutamine transporter GNP1 gene (38) is PCR amplified out of this fragment derived from the FTY720 resistant mutant or parent wild-type genomic DNA, then cloned into the same plasmid vector pRS416. The results of JK9-3dα transformants with these low-copy-number-plasmid DNA clearly shows that the anti-fungal effect of FTY720 is not inhibited by wild-type GNP1 gene, but mutant-derived GNP1 confers resistance to 30 μM of FTY720. The sequence analysis of GNP1 gene indicates that Trp 239 replaces Leu by point mutation in FTY720-resistant mutant yeast.

Only VAP1 (and TAP2) are isolated as genes, which confer FTY720 resistance In low-copy-number plasmid, from the other mutants-derived genomic DNA libraries Including wild-type one. These results suggest the possibility that the mutation happens In VAP1 (or TAP2) gene including non-coding region of this gene, resulting in the enhancement of the activity/function of these genes, although the mutation in the coding region of VAP1, at least, derived from FTY720 resistant mutants could not be found.

Analysis of FTY720 Resistant Recessive Mutant

Twenty one of 22 recessive mutants isolated are classified into 5 complementation groups (Group A: 13, Group B, C, D. E: each 2) by genetic crosses to each other opposite mating type mutant yeast (10 MH272-1 da mutants X 11 JK9-3dα mutants). The results suggest that 13 of 21 recessive mutant yeast have genetic mutation in the same gene. Therefore, 1 recessive spontaneous mutant yeast of 13, which belongs to group A, is chosen to identify the responsible mutant gene for conferring FTY720 resistance. The identification of mutant gene is performed by screening of wild type gene which complements the phenotype (resistance) of the recessive mutant yeast. More precisely, the recessive mutant is transformed with wild-type JK9-3dα genomic library in low-copy-number plasmid and FTY720 sensitivity-conferring gene is isolated.

Only one genomic DNA fragment in pRS416 confers sensitivity to FTY720. This DNA contains 4 intact genes (NRC1, Bul1, RCE1, DSK2). Then, the sensitivity of nrc1-, Bul1-, rce1- or dsk2-disrupted yeast strains to FTY720 Is tested and it is showed that only the Bull disruption confers resistance to 30 μM of FTY720. The introduction of Bul1 gene (encoding ubiquitin ligase binding protein) in low-copy-number plasmid pRS416 to Bull-disrupted yeast reverses the phenotype from resistance to sensitivity to FTY720. From these results, it is concluded that Bul1 gene of 4 genes located in DNA fragment conferring sensitivity to FTY720 complements the recessive mutation. Intact Bul1 gene in low-copy-number plasmid confers sensitvity to FTY720 in the resistant recessive mutants, implying that the mutation happens in Bul1 gene in the recessive mutant yeast. The results of DNA analysis indicates that Bul1 gene is deleted in the mutant strain.

These results not only indicate that Bul1 is required for the growth-inhibitory effects of FTY720, but also shows that ubiquitin ligase pathway, in which Bul1 is involved may play an important role on FTY720 action, since Bul1 is a binding protein of ubiquitin ligase Rsp5.

Constructon of DNA Libraries to Identify Mutant Genes from FTY720 Resistant Genomic Mutants

Total genomic DNA from dominant resistant mutants as well as parent yeast strain JK9-3dα is Isolated using Genomic-tip System (QIAGEN) according to standard methods. Then, DNA is subjected to partial Sau3AI and fragments are fractionated In continuous sucrose gradient. Following, 10-20 kb DNA fragments are collected and is ligated into the BamHI site of URA3-based low (CEN)-copy-number plasmid DNA pRS416. The ligation mixture is transformed into E.coli strain TOP10F′ (Invitrogen), >1×10⁵ Independent transformants are pooled, and plasmid DNA is isolated. The mean insert size of libries is 7-10 kb.

Cloning of Dominant Mutant Gene GNP1 Which Confers Resistance to FTY720

JK9-3dα wild-type cells are transformed with the yeast genomic DNA library constructed from FTY720 resistant dominant mutant in the low-copy-number vector pRS416. FTY720-resistant transformants are selected in SD medium in the presence of 30 μM of FTY720 as mentioned above. Plasmid inserts are sequenced and gene is identified as GNP1.

Cloning of Genomic DNA Fragment to Complement Recessive Mutation Conferring Resistant to FTY720

FTY720 resistant recessive mutant is transformed with wild-type genomic DNA library In pRS416 and URA3+transformants are selected on minimal plates lacking uracil at 30° C. Then, 8,000 independent transformants are picked up and FTY720-sensitive cells are selected on minimal plates in the presence of 30 μM of FTY720 at 19° C. to maintain plasmid DNA. Plasmid DNA are isolated from FTY720 sensitive transformants, amplified in E. coli and reintroduced into recessive resistant mutant for the confirmation of FTY720 sensitivity. The both terminii of FTY720 sensitive-conferring DNA insert is sequenced. Bul1 gene is identified as the complementary gene for the FTY720 resistant recessive mutation and the mutation of Bul1 gene is confirmed in the recessive mutant by the determination of DNA sequence.

The intact Bul1 gene is amplified by PCR from wild-type genomic DNA using primers ggAAgAATTCATTgTTCTTTCCCTTCAgCg and TTCgTCTAgAAAAgAgCACCAgAAA-AtgCA, purified, digested with EcoRI/XbaI, and cloned in pRS416.

Ubiquitin Ligase Rsp5 is Also Necessary for the Anti-Fungal Effects of FTY720

In order to clarify the role of the ubiquitin pathway in FTY720 action, the sensitivity of Rsp5 (Bul1 binding ubiquitin ligase) mutant to FTY720 is tested. Rsp5 mutant, in which Rsp5 expression level is reduced to less than 10% of wild-type due to the Ty1 transposed insertion in the promoter, is used in this study, since the complete disruption of Rsp5 gene is lethal. This Rsp5 mutant is resistant to 30 μM of FTY720 like Bul1 disrupted-yeast. This result supports that Bul1-Rsp5 targets and the ubiuquitin pathway play a pivotal role on the anti-fungal effects of FTY720.

The Involvement of Nutrients on the Anti-Fungal Effects of FTY720

The involvement of the amino acid transporter(s) in the anti-fungal effects of FTY720 is suggested from the findings that amino add transporters are isolated from the both approach, multicopy suppressers and dominant genomic mutants. If so, yeast that are able to synthesize amino acids might have a growth advantage in the presence of FTY720 compared with isogenic auxotrophic yeast that require amino acid import from the medium to grow. Therefore, the influence of nutrient auxotrophies present in JK9-3dα (trp1 his4 leu2 ura3), which are used to identify target molecules of FTY720, on the sensitivity to this compound is tested. In contrast to the parental auxotrophic strain, fully prototrophic TRP1 HIS4 LEU2 URA3 derivative of JK9-3dα is resistant to 30 μM of FTY720. However, prototrophic yeast is still sensitive to 100 μM. This result is consistent with that the multiple copies of VAP1 fail to overcome the growth inhibition by 100 μM FTY720. It therefore seems that the molecular targets and pathways other than that of 30 μM are involved in the effects of 100 μM of FTY720. Then, in order to identify which nutrient transport is inhibited by 30 μM of FTY720, prototrophic strain for each nutrient are constructed in JK9-3dα. Neither of each TRP1, HIS4, LEU2 or URA3 prototrophic strain is resistant to FTY720. However, the concomitant presence of both gene TRP1 and LEU2 is sufficient to confer resistance to 30 μM of FTY720 resistance, in contrast to the other combination. These results indicate that nutrient auxotrophies are required for growth inhibition by FTY720 (30 μM); FTY720 inhibits cell growth by impairing the transport of nutrients, especially tryptophan and leucin under these conditions of testing.

The amino acid transporter VAP1 has been cloned by screening genes that confer resistance to FTY720 in the high-copy-number plasmid. VAP1 has been reported to be amino acid transporter that imports valine, leucine, isoleucine, tyrosine, histidine and tryptophan. The anti-fungal effects of FTY720 are impeded by prototrophy for the concomitant TRP1 and LEU2. Furthermore, by means of the compensatory approach, the dissection of FTY720 resistant dominant mutants, it is showed that mutation of amino acid transporter GNP1 confers dominant FTY720-resistant phenotype. GNP1 has been reported to be high affinity transporter for glutamine and wild-type GNP1 is not likely to play an important role for tryptophan import, because no significant differences in the uptake rate of tryptophan are detected between cells that either lack or over-express GNP1. This is consistent with that GNP1 is not isolated as multicopy suppressers for overcoming FTY720 resistance. Incidentally, GNP1 shows the strong similarity to VAP1, they are 47.8% identical in the protein level. This similarity between them implies that substrate-specificity of GNP1 can possibly be changed by the point mutation and mutant GNP1 (W239L) gets ability to import amino acids other than glutamine, such as tryptophan and leucine, like VAP1. In addition to VAP1, wild type TAT2 gene In low-copy number-plasmid overcomes the anti-fungal effects of FTY720. TAT2 is so far known as a high affinity transporter for tryptophan. However, TAT2 may function as the transporter for the uptake of not only tryptophan, but also leucine in some extent, since prototrophy for tryptophan only is not sufficient to relieve anti-fugal effects of FTY720 and prototrophy for both tryptophan and leucine is required. TAT2 protein shows 39.7% identity with VAP1. FTY720 shows the anti-fungal effects on auxotrophic strains, which require amino acid import for cell survival, by impairing the import of amino acids such as tryptophan and leucine.

Furthermore, it is noteworthy that phytoshingosine (PHS) also diminishes tryptophan uptake and starves yeast cells for this amino acid. The growth-inhibitory effects of PHS are relieved by over-expression of VAP1 or TAT2, although the precise mode of PHS action remains to be elucidated. PHS is a sphingoid long chain base synthesized by yeast itself and structurally similar molecule to FTY720.

The above results of recessive mutants and gene disruption experiments indicate that FTY720 action is dependent on Bul1-Rsp5 ubiquitin-protein ligase. Because, 1) it is revealed that a responsible gene that confers the recessive FTY720 resistant phototype in the genomic mutant strain is Bul1 gene, a ubiquitin ligase Rsp5 binding protein. 2) The requirement of Bul1 for the FTY720 action is confirmed using complete Bull-disrupted yeast strain. Furthermore, 3) the anti-fungal effects of FTY720 is relieved by Rsp5 mutation which decreases the expression level of this gene. Bul1 itself is not essential for cell survival in contrast to Rsp5. However, the binding of Bul1 to Rsp5 is very important for yeast growth under various stress condition such as heat, salts and non-fermentable carbon source and the Bull-Rsp5 complex is required for decreasing the accumulation of toxic proteins produced upon various stress. Therefore, it seems that Bul1 is involved in the ubiquitination of a substrate portion catalyzed by Rsp5. Ubiquitin ligase has been implicated as playing an essential role in recognition for ubiquitination of the substrate. It is known that Rsp5 regulates the turnover of some membrane proteins such as Gap1 general amino transporters.

FTY720 accelerates endocytosis and degradation of nutrient transporter(s) dependent on Bul1-Rsp5-mediated ubiquitination pathway, resulting in the growth inhibition of yeast by starvation of nutrients such as leucine and tryptophan. Importantly, multiple copies of a putative deubiquitinating enzyme UBP11, which is also involved in ubiquitin-dependent protein turnover pathway like Rsp5 and Bul1, confers resistance to FTY720 as well as that of amino acid transporter VAP1. Although physiological function of UBP11 still remains undefined, the overexpression of UBP11 can negatively regulate the ubiquitination and turnover of the specific substrates (amino acid transporters), and leads to the increase of the amount of transporters that import amino acids such as tryptophan and leucine. Rsp5 protein mediated ubiquitination has been shown to be required for endocytosis of the uracil transporters in Saccharomyces cerevisiae.

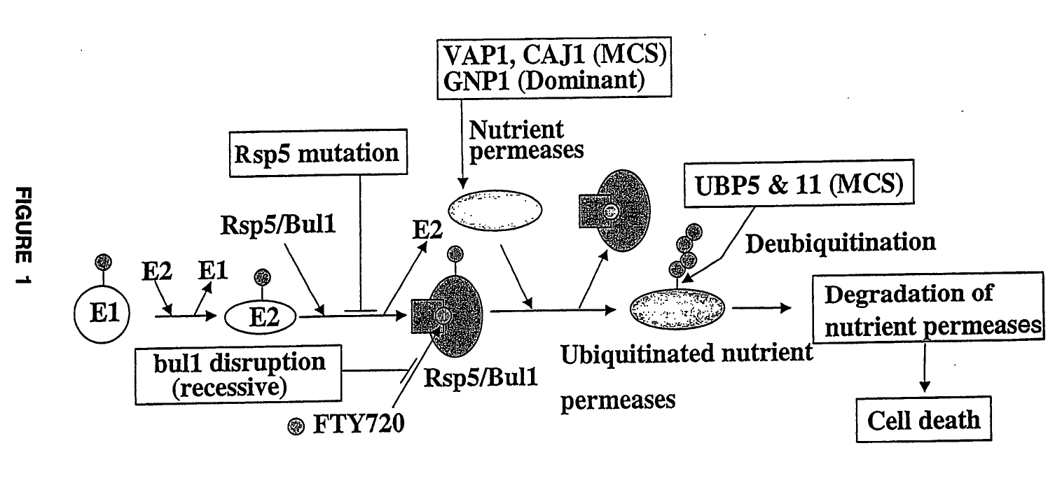

Based on above results, the mechanism of action of FTY720 on the inhibition of eukaryote cell growth is summarized in FIG. 1. The absolute presence of Bul1 and high concentrations of Rsp5 seem to be required for the growth sensitvity of FTY720 in this eukaryotic cell. It is interesting to note that while the disruption of Bul1 gene per se is not lethal for the growth of yeast strain, its presence is absolutely required for the growth inhibition in presence of FTY720. Similarly reduction in cellular concentrations of Rsp5 protein also seem to overcome FTY720 growth sensitivity. Based on these observations, it can be said that FTY 720 accelerates specific protein turnover in eukaryotic cells. Such accelerated protein turnover by FTY 720 can have significant physiological effects on cells. It is well known that several cell surface receptors including that of T-cells, e.g. G Protein-coupled receptor kinase, e.g EDG receptors, are turned over by ubiquitin mediated protein degradation. The acceleration of such protein degradation may make cells less responsive to interacting antigen presenting cells and thus result in immunosuppression. Alternately it is also known that specific protein kinases eg. GRK2, which down regulate chemokine receptor concentration on cell surface are turned over by ubiquitination. Acceleration of degradation of such type of proteins by FTY 720 can result in higher concentration of chemokine receptors eventually resulting in enhanced homing to lymph nodes. Ubiquitin pathways are well conserved between mammalian cells and yeast. The basic pathways are highly homologous and accordingly the target proteins involved in these pathways are well. Since there are Rsp5 mammalian homologues such as Nedd4 and Itch which are ubiquitin ligase proteins and since a ubiquitin ligase Itch-dependent proteolysis is an important mediator of the Immune response, it is likely that FTY720 has a similar mechanism of action on the inhibition of mammalian cells through this pathway. Alternatively it could be due to the ubiquitin mediated enhanced turnover of negative regulators of T-cell homing to lymph nodes. Overall, it indicates that FTY 720 mediated influence on enhanced ubiquitination and turnover of specific proteins that causes cellular effects Including immune suppression.

Similar results can be obtained with Compound Y.

In accordance with the foregoing, the present Invention provides:

1. Use of a compound of formula I, e.g. FTY720, or a pharmaceutically acceptable salt thereof, as a general modulator of protein expression through the ubiquitin pathway.

2. Use of a compound of formula I, e.g. FTY720, or a pharmaceutically acceptable salt thereof, as an inhibitor of eukaryote cell growth, e.g. of yeast cells.

3. A method for treating a pathogenic fungal infection in a mammalian subject comprising the step of administering to the subject a compound of formula I, e.g. FTY720, or a pharmaceutically acceptable salt thereof.

4.1 A method for modulating protein expression through the ubiquitin pathway in a medium, comprising adding a compound of formula I, e.g. FTY720, or a pharmaceutically acceptable salt thereof, to this medium.

4.2 A method for modulating protein expression in a subject, comprising treating the subject with an effective amount of a compound of formula I, e.g. FTY720, or a pharmaceutically acceptable salt thereof.

Preferred proteins are those which are degraded through the ubiquitin pathway.

5.1 Use of an ubiquitin pathway target, e.g. a ligase, e.g. Rsp5 or a ubiquitin ligase analogous to Rsp5, or a ligase binding protein, e.g. Bul1 or a ligase binding protein analogous to Bul1, as a target for immunosuppression.

5.2 A method for Inducing immunosuppression in a subject through an ubiquitin pathway target, comprising administering to the subject a compound which interferes with an ubiquitin pathway target, e.g. a ligase, e.g. Rsp5 or a ligase analogous to Rsp5, or a ligase binding protein, e.g. Bul1 or a ligase binding protein analogous to Bul1.

By “analogous” is meant a functionally equivalent variant of the ligase or the ligase binding protein that retains the properties of Rsp5 or Bul1, respectively. A protein is a variant of Rsp5 or Bul1 if it is at least 50%, preferably at least 70%, more preferably at least 80%, or particularly at least 90% homologous to said protein. Examples of a ligase analogous to Rsp5 include e.g. Nedd4 and Itch.

6.1 Use of a compound of formula I, e.g. FTY720, or a pharmaceutically acceptable salt thereof as an inhibitor of amino acid transport in T-cells.

6.2 Use of an inhibitor of amino acid transport in T-cells, e.g. a compound of formula I, e.g. FTY720, or a pharmaceutically acceptable salt thereof to inhibit T-cell replication or activation.

6.3 A method of inhibiting T-cell replication or activation in a subject comprising administering to the subject a compound which is capable of inhibiting amino acid transport in T-cells, e.g. a compound of formula I, e.g. FTY720 or a pharmaceutically acceptable salt thereof.

7.1 Use of a compound of formula I, e.g. FTY720, or a pharmaceutically acceptable salt thereof, in any of the methods above.

7.2 Use of a compound of formula I, e.g. FTY720, or a pharmaceutically acceptable salt thereof, in the preparation of a medicament for use in any of the methods 3,4.2, 5.2 or 6.3.

8. Use of a compound of formula I, e.g. FTY720, or a pharmaceutically acceptable salt thereof, to downregulate G Protein coupled receptor kinase, e.g EDG receptors.

Daily dosages required in practicing the method of the present invention will vary depending upon, for example the host, the mode of administration, the severity of the condition to be treated. A preferred daily dosage range is about from 0.03 to 2.5 mg/kg per day as a single dose or in divided doses. Suitable daily dosages are on the order of from e.g. 0.5 to 50 mg p.o. Suitable unit dosage forms for oral administration comprise from ca. 0.1 to 25 mg compound of formula I, e.g. FTY720, e.g. in hydrochloride form, together with one or more pharmaceutically acceptable diluents or carriers therefor. As an alternative, the compound of formula I, e.g. FTY720, may also be administered twice or three times a week, e.g. at a dosage as indicated above.

Compound of formula 1, e.g. FTY720, may be administered by any conventional route, in particular enterally, e.g. orally, for example in the form of solutions for drinking, tablets or capsules or parenterally, for example in the form of injectable solutions or suspensions. Pharmaceutical compositions comprising a compound of formula I may be manufactured in conventional manner, e.g. as described in EP-A1-627,406 or EP-A-1,002,792.

Compound of formula I may be administered as the sole active ingredient or together with an active co-agent, e.g. an immunomodulating drug, an anti-inflammatory agent or an anti-bacterial or anti-viral agent.

Where the compound of formula I is administered in conjunction with e.g. other immunosuppressive/immunomodulatory, anti-inflammatory, anti-bacterial or anti-viral therapy, dosages of the co-administered immunosuppressant, immunomodulatory, anti-inflammatory, anti-bacterial or anti-viral compound will of course vary depending on the type of co-drug employed, on the condition being treated and so forth. The co-agent may be administered by any conventional route, in particular enterally, e.g. orally, e.g. in the form of tablets, capsules, drink solutions, nasally, pulmonary (by inhalation) or parenterally, e.g. in the form of injectable solutions or suspensions.

In accordance with the foregoing the present invention provides in a yet further aspect:

9. A method as defined above comprising co-administration, e.g. concomitantly or in sequence, of a therapeutically effective amount of a compound of formula I or a pharmaceutically acceptable salt thereof, and a second drug substance, said second drug substance being an immunosuppressant, immunomodulatory, anti-inflammatory, anti-bacterial or anti-viral drug.

10. A therapeutic combination, e.g. a kit, e.g. for use in any method as defined above, comprising a compound of formula I, or a pharmaceutically acceptable salt thereof, to be used concomitantly or in sequence with at least one pharmaceutical composition comprising an immunosuppressant, immunomodulatory, anti-inflammatory, anti-bacterial or anti-viral drug. The kit may comprise instructions for its administration.

1. (canceled)

2. (canceled)

3. A method for treating a pathogenic fungal infection in a mammalian subject comprising the step of administering to the subject a compound of formula I 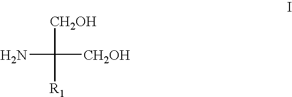
wherein R₁ is 2-(4-octylphenyl)ethyl or 2-[4-(1-oxo-5-phenylpentyl)phenyl]ethyl, or a pharmaceutically acceptable salt thereof.

4. A method for modulating protein expression through the ubiquitin pathway in a medium, comprising adding a compound of formula I 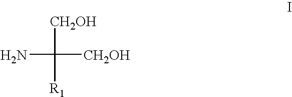
wherein R₁ is 2-(4-octylphenyl)ethyl or 2-[4-(1-oxo-5-phenylpentyl)phenyl]ethyl or a pharmaceutically acceptable salt thereof, to this medium.

5. A method for modulating protein expression in a subject, comprising treating the subject with an effective amount of a compound of formula I 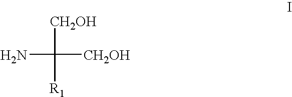
wherein R₁ is 2-(4-octylphenyl)ethyl or 2-[4-(1-oxo-5-phenylpentyl)phenyl]ethyl or a pharmaceutically acceptable salt thereof.

6. (canceled)

7. A method for inducing immunosuppression in a subject through an ubiquitin pathway target, comprising administering to the subject a compound which interferes with an ubiquitin pathway target.

8. (canceled)

9. (canceled)

10. A method of inhibiting T-cell replication or activation in a subject comprising administering to the subject a compound which is capable of inhibiting amino acid transport in T-cells.

11. (canceled)

12. The method of claim 7 wherein said compound is the compound of formula I 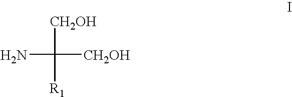
or a pharmaceutically acceptable salt thereof.

13. The method of claim 10 wherein said compound is the compound of formula I 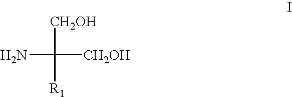
or a pharmaceutically acceptable salt thereof.

14. The method of claim 3 wherein the amount of the compound of formula I administered is 0.03 to 2.5 mg/kg per day.

15. The method of claim 3 wherein the compound of formula I is administered in conjunction with a second drug substance.

16. The method of claim 14 wherein said second drug substance is an immunosuppressant, immunomodulatory, anti-inflammatory, anti-bacterial or anti-viral drug.

17. The method of claim 4 wherein the amount of the compound of formula I administered is 0.03 to 2.5 mg/kg per day.

18. The method of claim 4 wherein the compound of formula I is administered in conjunction with a second drug substance.

19. The method of claim 18 wherein said second drug substance is an immunosuppressant, immunomodulatory, anti-inflammatory, anti-bacterial or anti-viral drug.

20. The method of claim 5 wherein the amount of the compound of formula I administered is 0.03 to 2.5 mg/kg per day.

21. The method of claim 5 wherein the compound of formula I is administered in conjunction with a second drug substance.

22. The method of claim 21 wherein said second drug substance is an immunosuppressant, immunomodulatory, anti-inflammatory, anti-bacterial or anti-viral drug.

23. The method of claim 7 wherein the amount of the compound of formula I administered is 0.03 to 2.5 mg/kg per day.

24. The method of claim 7 wherein the compound of formula I is administered in conjunction with a second drug substance.

25. The method of claim 24 wherein said second drug substance is an immunosuppressant, immunomodulatory, anti-inflammatory, anti-bacterial or anti-viral drug.

26. The method of claim 10 wherein the amount of the compound of formula I administered is 0.03 to 2.5 mg/kg per day.

27. The method of claim 10 wherein the compound of formula I is administered in conjunction with a second drug substance.

28. The method of claim 27 wherein said second drug substance is an immunosuppressant, immunomodulatory, anti-inflammatory, anti-bacterial or anti-viral drug.
